(19) United States
(12) Patent Application Publication
(10) Pub. No.: US 2018/0042833 A1
(43) Pub. Date: Feb. 15, 2018

(54) Cleaning Cream Containing PVA Particles and Producing Method Thereof

(71) Applicants: XING YAN Plastic Co., Ltd., Xiushui Township (TW); Jyun He Technology Limited Company, Zhunan Township (TW)

(72) Inventors: Hong-Chang Yan, Xiushui Township (TW); You-Hong Lu, Zhunan Township (TW); Fang-Yun Lin, Zhunan Township (TW)

(21) Appl. No.: 15/668,757

(22) Filed: Aug. 4, 2017

(30) Foreign Application Priority Data
Aug. 11, 2016 (TW) 105125532

(51) Int. Cl. A61K 8/81; A61K 8/34; A61Q 19/10

(52) U.S. Cl. CPC A61K 8/8135; A61Q 19/10; A61K 8/345; A61K 2800/28; A61K 2800/51

(57) ABSTRACT

A cleaning cream containing PVA particles has PVA particles being uniformly dispersed in a cleaning cream. Moisture content of the cleaning cream is at the range of 0.1˜23%. The cleaning cream comprises a least a surfactant. The PVA particles has glycerol and PVA in which molecular weight of the PVA is 110,000˜120,000. The PVA particles remain as particle form in the cleaning cream.

### BACKGROUND OF THE INVENTION

### 1. Field of the Invention

The present invention relates to a cleaning cream, and more particularly to a cleaning cream containing PVA particles.

### 2. Description of Related Art

Cleaning products that use for exfoliating or whitening skin and teeth are normally contained tiny plastic particles made of Polyethylene (PE) or Polypropylene (PP). When applying the cleaning products, the PE or PP made plastic particles are able to remove dead skin or teeth tartar by rubbing user's skin or teeth.

Because of above mentioned advantages of the PE or PP made plastic particles, more and more applications are introduced to the market. As time goes by, the PE or PP made plastic particles are secretly damaging our health and our treasure planet.

In order to achieve better function and texture, the PE or PP made plastic particles are usually produced with tiny size and added into cleaning products in great deal amount. According to the statistics, at least three hundred and sixty thousands of tiny PE or PP made plastic particles are added into a bottle of facial cleaning cream. It means that tens of thousands of the tiny PE or PP made plastic particles are drained into sewers with contaminated water. Since the PE or PP made plastic particles are too tiny to be collected by water purification plant, this PE or PP made plastic particles will enter into the ocean and our environment eventually. The non-degradable PE or PP made plastic particles then become food for sea creatures or animals and remain in its body for lifespan. After eating this contaminated sea creatures or animals, the PE or PP made plastic particles will transfer to human body and affect human health.

Although some grained natural fiber were used as replacement, there still some limitations like high cost and difficulty of controlling particle size lead grained natural fiber hard to be wildly used as replacement.

To overcome the shortcomings, the present invention provides a cleaning cream containing PVA particles to mitigate or obviate the aforementioned problems.

### SUMMARY OF THE INVENTION

The main objective of the present invention is to provide a cleaning cream containing PVA particles comprising: PVA particles being uniformly dispersed in a cleaning cream. The cleaning cream comprises at least a surfactant and the moisture content of the cleaning cream is at the range of 0.1˜23 wt %. The PVA particles comprise PVA 50˜95 wt % and glycerine 0.5˜50 wt % in which molecular weight of PVA is ranging from 110,000 to 120,000 Dalton. The PVA particles remain in particle form permanently in the cleaning cream.

PVA particles of the present invention are able to stay as particle form in the cleaning cream that contained certain quantity of water. The present invention may provide excellent exfoliation and whitening functions when applied to human body. After draining into sewers with contaminated water, the PVA particles of the present invention will be degraded or dissolved by great amount of water which may solve the problems that conventional PE or PP made plastic particles are not degradable in the environment.

The PVA particles of the present invention may be manufactured by conventional plastic processing process. The cost will be low and the particle size will be easily controlled by the present invention.

Other objectives, advantages and novel features of the invention will become more apparent from the following detailed description when taken in conjunction with the accompanying drawings.

### BRIEF DESCRIPTION OF THE DRAWINGS

FIG. 1 is a flow chart for producing PVA particles in accordance with the present invention.

### DETAILED DESCRIPTION OF THE PREFERRED EMBODIMENT

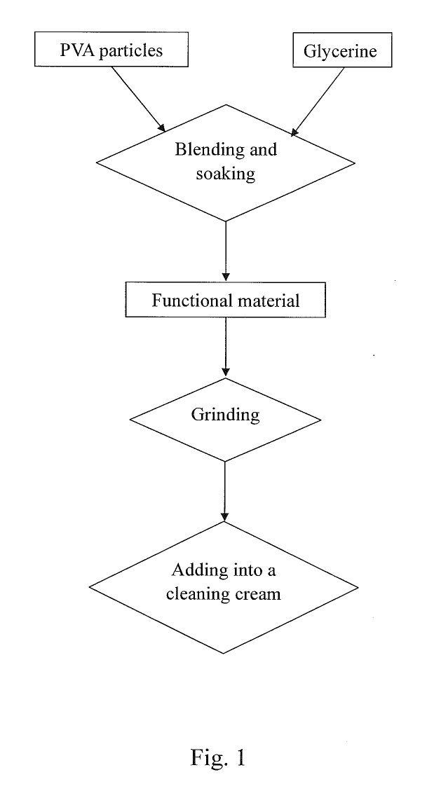

With reference to FIG. 1, a first embodiment for producing cleaning cream containing PVA (Polyvinyl Acetate) particles of the present invention comprises steps of:

blending PVA particles and glycerine by a high speed mixer letting PVA particles being completely soaked with the glycerine, wherein the PVA particles contain PVA 50˜95 wt % and glycerine 0.5˜50 wt % after soaking with the glycerine;

adding a functional material 0.5˜4.5 wt % while blending;

grinding the PVA particles to smooth its surfaces; and mixing the PVA particles with a cleaning cream which the moisture content of the cleaning cream is at the range of 0.1˜23 wt % and the cleaning cream contained at least a surfactant. The PVA particles mixing in the cleaning cream are preferred at range of 0.5˜5 wt %.

Particle size of unprocessed PVA particle is around 100 mesh (150 μm) which is incredibly hard and having sharp edges on its surface. After soaking with the glycerine, the particle size of PVA particles will increase to 70˜80 mesh (180˜220 μm) and become much softer and smoother. This may provide comfort and better quality when applied to human body without causing any scratch or even injury.

The functional material as mentioned above may be but not limited to colorant, fragrant or essence. The cleaning cream of the present invention may be a cream, gel or paste. The surfactant in the cleaning cream may be but not limited to anionic surfactant, cationic surfactant, nonionic surfactant or ampholytic surfactant. Beside the surfactant, the cleaning cream may further contain additives as desired like chelating agent or preservative.

PVA is water soluble polymer. It will be dissolved or degraded in an environment contained great amount of water or moisture. In order to keep the PVA particles as complete particle form in the cleaning cream contained with water/moisture, the molecular weight of PVA of the present invention is preferred at the range of 110,000˜120,000 D (Dalton, D). As the molecular weight increased, the viscosity of PVA becomes higher and the water soluble property of PVA otherwise becomes lower. Hence, the viscosity, water-solubility and texture of PVA in the present invention may be controlled by using PVA with different molecular weights.

On the other hand, PVA with molecular weight at the range of 110,000˜120,000 D has better absorption ability to the glycerine which will end up with products with excellent quality and texture. The PVA particles of the present invention are able to maintain stable in the cleaning cream. When applied to human body, the PVA particles of the present invention are able to remove dead skin or teeth tartar without causing any scratch or injury. After draining to the sewer, the PVA particles will be dissolved or degraded by water which won't cause any damage or contamination to the ocean or the environment.

The cleaning products include kinds of preparations with different use methods. To apply directly on the skin or teeth and to apply by forming foam with adding a little bit of water are two most commonly use methods. The foam will contain water/moisture up to 50˜60 wt % which is higher than the cleaning cream of the present invention (23 wt %). The PVA particles of the present invention is still able to keep as complete particle form for at least 2˜5 minutes to maintain its function. This may credit to the molecular weight of the PVA as mentioned above.

Main materials of the present invention only contain PVA and glycerine. These two materials are considered eco-friendly and safe to human body. It will also cause no harm to sea creatures or animals even being discharge to the ocean or environment.

With reference to below chart 1, samples of the present invention are provided after produced by above mentioned producing method. Chart 1 shows the content of glycerine in PVA particles, expanded size of PVA particles and appearance and texture of PVA particles after soaking with glycerine.

CHART 1

| | Glycerine | Expanded size | |
|---|---|---|---|
| | (wt %) | (times) | Appearance and texture |
| Sample 1 | 20 | 4 times bigger | Appearance: round, small |
| | | than unprocessed | Texture: hard |
| | | PVA particles | Application: Suitable for the |
| | | | cleaning cream like toothpaste |
| | | | or exfoliating product for foot. |
| Sample 2 | 30 | 6 times bigger | Appearance: round, moderate |
| | | than unprocessed | Texture: moderate |
| | | PVA particles | Application: Suitable for most |
| | | | cleaning creams. |
| Sample 3 | 35 | 7 times bigger | Appearance: round, big |
| | | than unprocessed | Texture: soft |
| | | PVA particles | Application: Suitable for the |
| | | | cleaning cream like |
| | | | exfoliating or whitening |
| | | | products for face or body. |

According to description above, PVA particles of the present invention are able to stay as particle form in the cleaning cream that contained certain quantity of water. The present invention may provide excellent exfoliation and whitening functions when applied to human body. After draining into sewers with contaminated water, the PVA particles of the present invention will be degraded or dissolved by great amount of water which may solve the problems that conventional PE or PP made plastic particles are not degradable in the environment.

The PVA particles of the present invention may be manufactured by conventional plastic processing process. The cost will be low and the particle size will be easily controlled by the present invention.

What is claimed is:

1. A cleaning cream containing PVA particles comprising:
polyvinyl acetate particles being uniformly dispersed in a cleaning cream;
the cleaning cream having at least a surfactant and a moisture content of the cleaning cream being at the range of 0.1˜23 wt %;
the polyvinyl acetate particles being mixed in the cleaning cream at a range of 0.5˜5 wt %;
the polyvinyl acetate particles having polyvinyl acetate 50˜95 wt % and glycerine 0.5˜50 wt % in which molecular weight of polyvinyl acetate being ranging from 110,000 to 120,000 Dalton; and
the polyvinyl acetate particles remaining in particle form in the cleaning cream.

2. The cleaning cream containing PVA particles as claimed in claim 1, wherein the cleaning cream contains chelating agent or preservative.

3. The cleaning cream containing PVA particles as claimed in claim 1, wherein the surfactant is anionic surfactant, cationic surfactant, nonionic surfactant or ampholytic surfactant.

4. The cleaning cream containing PVA particles as claimed in claim 1, wherein the polyvinyl acetate particles contain a functional material 0.5˜4.5 wt %.

5. The cleaning cream containing PVA particles as claimed in claim 4, wherein the functional material is colorant, fragrant or essence.

6. A producing method of a cleaning cream containing PVA particles comprising steps of:
blending polyvinyl acetate particles and glycerine by a high speed mixer letting polyvinyl acetate particles being completely soaked with the glycerine, wherein the polyvinyl acetate particles contain polyvinyl acetate 50˜95 wt % and glycerine 0.5˜50 wt % after soaking with the glycerine;
adding a functional material 0.5˜4.5 wt % while blending;
grinding the PVA particles to smooth its surfaces;
mixing the polyvinyl acetate particles with a cleaning cream which a moisture content of the cleaning cream is at the range of 0.1˜23 wt % and the cleaning cream contained at least a surfactant; and
the PVA particles being mix in the cleaning cream at a range of 0.5˜5 wt %.
